(19) United States
(12) Patent Application Publication
(10) Pub. No.: US 2022/0249758 A1
(43) Pub. Date: Aug. 11, 2022

(54) BLOOD CLEANING DEVICE

(71) Applicant: Dongxu Zhang, Guangzhou (CN)

(72) Inventor: Dongxu Zhang, Guangzhou (CN)

(21) Appl. No.: 17/375,002

(22) Filed: Jul. 14, 2021

(30) Foreign Application Priority Data
Feb. 8, 2021 (CN) 202120350456.6

(51) Int. Cl. A61M 1/36; A61M 39/22; B01D 53/04

(52) U.S. Cl. CPC A61M 1/3621; B01D 53/0438; B01D 53/0454; A61M 39/22

(57) ABSTRACT

The invention discloses a blood cleaning device, comprising a first hyperbaric oxygen ring-shaped container, a second hyperbaric oxygen ring-shaped container, an adsorption particle ring-shaped container, and a control panel; the first hyperbaric oxygen ring-shaped container is connected with an original blood inlet pipe; the second hyperbaric oxygen hyperbaric container is provided with a cleaned blood outlet pipe; the cleaned blood outlet pipe is provided with an outlet valve; the first hyperbaric oxygen ring-shaped container and the adsorption particle ring-shaped container are connected by a first connecting pipe and a second connecting pipe; the adsorption particle ring-shaped container is connected to the second hyperbaric oxygen hyperbaric container by a third connecting pipe. The invention can clean the blood, reduce the substances harmful to the human body in the blood, achieve the purpose of improving the sub-health status of the human body, and has a good promotion prospect.

### BACKGROUND OF THE INVENTION

### 1. Field of the Invention

The invention relates to the technical field of blood cleaning, in particular to a blood cleaning device.

### 2. Description of the Related Art

Blood is important to overall health. It not only supplies oxygen to cells, but also transmits nutrients, helps eliminate garbage, controls body temperature, heals wounds and fights infections. Therefore, in order to stay healthy, it is recommended to clean your blood regularly, especially when you start to feel weak or sick. The cleaning process usually takes 3-5 days. There is no blood cleaning device on the market that has a good cleaning effect and is easy to use.

### SUMMARY OF THE INVENTION

The objective of the invention is to solve the problems in the prior art, and provide a blood cleaning device.

In order to solve the above technical problems, the invention provides the technical solutions as follows: a blood cleaning device, comprising a first hyperbaric oxygen ring-shaped container, a second hyperbaric oxygen ring-shaped container, an adsorption particle ring-shaped container, and a control panel, wherein the adsorption particle ring-shaped container is provided with adsorption particles; the first hyperbaric oxygen ring-shaped container is connected with an original blood inlet pipe; the second hyperbaric oxygen hyperbaric container is provided with a cleaned blood outlet pipe; the cleaned blood outlet pipe is provided with an outlet valve; the first hyperbaric oxygen ring-shaped container and the adsorption particle ring-shaped container are connected by a first connecting pipe and a second connecting pipe; the first connecting pipe is provided with a first connecting valve, and the second connecting pipe is provided with a second connecting valve; the adsorption particle ring-shaped container is connected to the second hyperbaric oxygen hyperbaric container by a third connecting pipe; the third connecting pipe is provided with a third connecting valve; the first connecting valve, the second connecting valve, the third connecting valve, and the outlet valve are all electrically connected to the control panel, and the first connecting valve, the second connecting valve, the third connecting valve, and the outlet valve are all controlled by the control panel.

Preferably, the original blood inlet pipe is provided with an inlet valve.

Preferably, the inlet valve is electrically connected to the control panel, and the inlet valve is controlled by the control panel.

Preferably, the control panel is an LCD control panel.

The invention has the following advantageous; it can clean the blood, reduce the substances harmful to the human body in the blood, achieve the purpose of improving the sub-health status of the human body, and has a good promotion prospect.

### BRIEF DESCRIPTION OF THE DRAWINGS

FIG. 1 is a schematic diagram of the structure of a blood cleaning device of the invention.

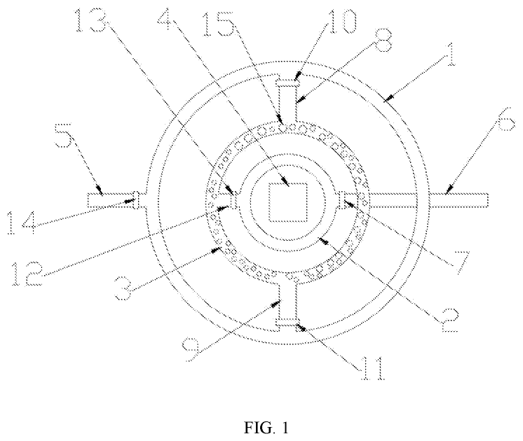

As shown in the FIGURE: 1 refers to the first hyperbaric oxygen ring-shaped container; 2 refers to the second hyperbaric oxygen ring-shaped containers; 3 refers to the adsorption particle ring-shaped container; 4 refers to the control panel; 5 refers to the original blood inlet pipe; 6 refers to the cleaned blood outlet pipe; 7 refers to the outlet valve; 8 refers to the first connecting pipe; 9 refers to the second connecting pipe; 10 refers to the first connecting valve; 11 refers to the second connecting valve; 12 refers to the third connecting pipe; 13 refers to the third connecting valve; 14 refers to the inlet valve; 15 refers to the adsorption particle.

### DETAILED DESCRIPTION OF THE PREFERRED EMBODIMENTS

In the description of the invention, it should be understood that the orientation or positional relationship indicated by the terms “center”, “longitudinal”, “transverse”, “length”, “width”, “thickness”, “upper”, “lower”, “front”, “back”, “left”, “right”, “vertical”, “horizontal”, “top”, “bottom”, “inner”, “outer”, “clockwise”, “counterclockwise” etc. are based on the orientation or positional relationship shown in the drawings, and is only for the convenience of describing the invention and simplifying the description, but not indicate or imply that the pointed device or element must have a specific orientation, be constructed and operated in a specific orientation, and therefore cannot be understood as a limitation of the invention.

In addition, the terms “first” and “second” are only used for descriptive purposes, and cannot be understood as indicating or implying relative importance or implicitly indicating the number of indicated technical features. Thus, the features defined with “first” and “second” may explicitly or implicitly include one or more of these features. In the description of the invention, unless otherwise clearly defined, “plurality” means two or more.

In order to facilitate the understanding of the invention, the invention will be described in detail hereinafter with reference to the drawings.

The preferred embodiments of the invention are shown in the drawings. However, the invention can be implemented in many different forms and is not limited to the embodiments described herein. On the contrary, the purpose of providing these embodiments is to make the understanding of the disclosure of the invention more thorough and comprehensive.

With reference to the drawing, a blood cleaning device, comprising a first hyperbaric oxygen ring-shaped container 1, a second hyperbaric oxygen ring-shaped container 2, an adsorption particle ring-shaped container 3, and a control panel 4, wherein the adsorption particle ring-shaped container 3 is provided with adsorption particles 15; the first hyperbaric oxygen ring-shaped container 1 is connected with an original blood inlet pipe 5; the second hyperbaric oxygen hyperbaric container 2 is provided with a cleaned blood outlet pipe 6; the cleaned blood outlet pipe 6 is provided with an outlet valve 7; the first hyperbaric oxygen ring-shaped container 1 and the adsorption particle ring-shaped container 3 are connected by a first connecting pipe 8 and a second connecting pipe 9; the first connecting pipe 8 is provided with a first connecting valve 10, and the second connecting pipe 9 is provided with a second connecting valve 11; the adsorption particle ring-shaped container 3 is connected to the second hyperbaric oxygen hyperbaric container 2 by a third connecting pipe 12; the third connecting pipe 12 is provided with a third connecting valve 13; the first connecting valve 10, the second connecting valve 11, the third connecting valve 13, and the outlet valve 7 are all electrically connected to the control panel 4, and the first connecting valve 10, the second connecting valve 11, the third connecting valve 13, and the outlet valve 7 are all controlled by the control panel 4. The original blood inlet pipe 5 is provided with an inlet valve 14. The inlet valve 14 is electrically connected to the control panel 4, and the inlet valve 14 is controlled by the control panel 4. The control panel 4 is an LCD control panel.

The working principle of the invention: the blood enters from the original blood inlet pipe, and the entry can be realized by controlling the inlet valve through the control panel; at this time, the first connecting valve, the second connecting valve, the third connecting valve, and the outlet valve are all closed; the blood flows in the first hyperbaric oxygen ring-shaped container for processing, then the first connecting valve and the second connecting valve are opened through the control panel, and the blood enters the adsorption particle ring-shaped container for processing; then the third connecting valve is opened, and the blood flows in the second hyperbaric oxygen ring-shaped container for processing; then the outlet valve is opened, and the blood after the cleaning process returns to the human body from the cleaned blood outlet pipe.

The technical features of the above embodiments can be combined arbitrarily. To make the description concise, all possible combinations of the technical features in the above embodiments are not described. However, as long as there is no contradiction in the combination of these technical features, it should be regarded as the scope described in the specification. The above embodiments only express several implementation modes of the invention, and their description is relatively specific and detailed, but they should not be understood as a limitation on the scope of the invention. It should be understood that several modifications and improvements may be made by those of ordinary skill in the art without departing from the concept of the invention, which shall all fall within the protection scope of the invention. Therefore, the protection scope of the invention shall be subject to the appended claims.

1. A blood cleaning device, comprising a first hyperbaric oxygen ring-shaped container, a second hyperbaric oxygen ring-shaped container, an adsorption particle ring-shaped container, and a control panel, wherein the adsorption particle ring-shaped container is provided with adsorption particles; the first hyperbaric oxygen ring-shaped container is connected with an original blood inlet pipe; the second hyperbaric oxygen hyperbaric container is provided with a cleaned blood outlet pipe; the cleaned blood outlet pipe is provided with an outlet valve; the first hyperbaric oxygen ring-shaped container and the adsorption particle ring-shaped container are connected by a first connecting pipe and a second connecting pipe; the first connecting pipe is provided with a first connecting valve, and the second connecting pipe is provided with a second connecting valve; the adsorption particle ring-shaped container is connected to the second hyperbaric oxygen hyperbaric container by a third connecting pipe; the third connecting pipe is provided with a third connecting valve; the first connecting valve, the second connecting valve, the third connecting valve, and the outlet valve are all electrically connected to the control panel, and the first connecting valve, the second connecting valve, the third connecting valve, and the outlet valve are all controlled by the control panel.

2. The blood cleaning device according to claim 1, wherein the original blood inlet pipe is provided with an inlet valve.

3. The blood cleaning device according to claim 2, wherein the inlet valve is electrically connected to the control panel, and the inlet valve is controlled by the control panel.

4. The blood cleaning device according to any one of claim 3, wherein the control panel is an LCD control panel.
